(19) United States
(12) Plant Patent Application Publication
(10) Pub. No.: US 2010/0212054 P1
(43) Pub. Date: Aug. 19, 2010

(54) Pennisetum 'Tift-23'

(50) ;

(75) Inventors: Wayne William Hanna, Chula, GA (US); S. Kristine Braman, Griffin, GA (US)

Correspondence Address: DAVIS WRIGHT TREMAINE LLP - San Francisco, 505 MONTGOMERY STREET, SUITE 800, SAN FRANCISCO, CA, 94111, US

(73) Assignee: University of Georgia Research Foundation, Inc., Athens, GA (US)

(21) Appl. No.: 12/378,553

(22) Filed: Feb. 17, 2009

(51) Int. Cl. A01H 5/00

(52) U.S. Cl. PLT/373

(57) ABSTRACT

The new variety Pennisetum ‘Tift-23’ is provided. The new and distinct variety has increased tillers under drought conditions; superior Helminthosporium leaf spot resistance; high ornamental value; narrow canopy; narrow base; and narrow leaves. The asexually reproduced inter-specific variety is reliably propagated vegetatively.

### LATIN NAME OF THE GENUS AND SPECIES OF THE PLANT CLAIMED

‘Tift-23’ is an inter-specific ornamental Pennisetum hybrid of the genus and species Pennisetum purpureum×Pennisetum glaucum.

### VARIETY DENOMINATION

The new Pennisetum claimed is of the variety denominated ‘Tift-23’.

### BACKGROUND OF THE INVENTION

The present invention relates to a new and distinct cultivar of Pennisetum botanically known as Pennisetum purpureum×Pennisetum glaucum, and herein referred to as ‘Tift-23’.

The new Pennisetum is a product of a planned breeding program conducted by the Inventors in Tifton, Ga. The objective of the Pennisetum breeding program is to create new plant cultivars with improved commercial qualities. This cultivar is commercially important for its superior ornamental value, since the plant (crown and base) and the leaves are narrower, giving it a fine-textured appearance. Moreover, it shows better pest resistance. These and other qualities are enumerated herein.

Pedigree and history: In 2003, napiergrass (unpatented, Pennisetum purpureum, 2n=4x=28, designated ‘N240-8’, the female parent) was crossed with red pearl millet (unpatented Pennisetum glaucum, the male parent). The new variety ‘Tift-23’ was selected in 2004 from the 2003 cross.

Asexual reproduction of the new Pennisetum by vegetative propagation (single stem propagules) in a controlled environment in Tifton, Blairsville and Griffin, Ga. since 2005, has shown that the unique features of this new Pennisetum are stable and reproduced true to type in successive generations.

### SUMMARY OF THE INVENTION

The new variety Pennisetum ‘Tift-23’ has not been observed under all possible environmental conditions. The phenotype may vary somewhat with variations in environment and cultural practices such as temperature and light intensity without, however, any variance in genotype.

The following traits have been repeatedly observed in Tifton, Blairsville, and Griffin Ga., and are determined to be the unique characteristics of the new variety ‘Tift-23’:

1. Superior tiller numbers under drought conditions;

2. Superior Helminthosporium leaf spot resistance;

3. High ornamental value;

4. Narrow canopy;

5. Narrow base;

6. Narrow leaves.

The new variety Pennisetum ‘Tift-23’ can be compared to its parents, as well as other grasses developed by University of Georgia researchers, such as the patented (U.S. Plant Pat. No. 18,509) Pennisetum ‘Prince’, patented (U.S. Plant Pat. No. 17,728) Pennisetum ‘Princess’, and co-pending plant patent application for ‘Tift-17’ (Attorney Docket No. 0088564-008US0), filed on the same date as the instant application.

Comparison to ‘N240-8’. The new variety ‘Tift-23’ is similar to ‘N240-8’ in the absence of trichomes on both leaf surfaces, sheath, and leaf blade margin trichomes. Both are perennials and neither flower in late October in Tifton, Ga. The new variety ‘Tift-23’ is shorter and has different leaf color (adaxial surface, abaxial surface and midrib) than ‘N240-8’. The new variety ‘Tift-23’ has shorter leaf length and narrower leaf width than ‘N240-8’. The new variety ‘Tift-23’ has slightly shorter trichomes on the leaf collar than ‘N240-8’. The new variety ‘Tift-23’ has approximately 2 mm long trichomes about 3 cm from collar on the leaf blade edge whereas ‘N240-8’ has slightly longer leaf blade edge trichomes, slightly farther from the collar. The new variety ‘Tift-23’ is not disease-susceptible to Helminthosporium leaf spot, whereas ‘N240-8’ is susceptible to this disease.

Comparison to red tetraploid millet. The new variety ‘Tift-23’ has similar abaxial leaf and midrib color to red millet. Neither the new variety ‘Tift-23’ nor red millet has adaxial or abaxial leaf surface, or sheath trichomes. The new variety ‘Tift 23’ is approximately 50% shorter than red millet. The new variety ‘Tift-23’ has shorter leaf length and narrower leaf width than red millet. The new variety ‘Tift-23’ has inconspicuous leaf blade margin trichomes, whereas red millet has slightly apparent leaf blade margin trichomes. The new variety ‘Tift-23’ has less dense and shorter leaf collar trichomes than red millet. The new variety ‘Tift-23’ has approximately 2 mm long trichomes about 3 cm from collar on the leaf blade edge whereas red millet has none. The new variety ‘Tift-23’ is a perennial at Tifton, Ga., whereas red millet is an annual at the same location. The new variety ‘Tift-23’ is not disease-susceptible to Piricularia grisea leaf spot, whereas red millet is susceptible to this disease. The new variety ‘Tift-23’ has no inflorescences in late October, in Tifton, Ga., whereas red millet does have inflorescences.

Comparison to ‘Prince’. The new variety ‘Tift-23’ is significantly shorter than ‘Prince’. It was significantly shorter than ‘Prince’ in 5 of 6 tests. Leaf length of the new variety ‘Tift-23’ is significantly shorter than that of ‘Prince’. The new variety ‘Tift-23’ rated a significantly higher ornamental value than ‘Prince’ in all tests and in two of three tests for color. Field trials indicate that the new variety ‘Tift-23’ has improved resistance to Helminthosporium leaf spot as compared to ‘Prince’.

Comparison to ‘Princess’. The new variety ‘Tift-23’ was significantly shorter than ‘Princess’ in four of seven tests. Canopy and plant base of the new variety ‘Tift-23’ tended to be narrower or similar to that of ‘Princess’. Leaves of the new variety ‘Tift-23’ were narrower in three of four tests and similar in length to that of ‘Princess. The number of tillers were similar for the new variety ‘Tift-23’ and ‘Princess’ except under drought stress conditions, when the new variety ‘Tift-23’ produced significantly more tillers. The new variety ‘Tift-23’ rated significantly better than ‘Princess’ for ornamental value at 2 of 3 locations and in 1 of 2 years for color. The new variety ‘Tift-23’ showed better resistance to Helminthosporium leaf spot than ‘Princess’ in two of three tests.

Comparison to co-pending ‘Tift-17’. ‘Tift-17’ is approximately twice as tall, has longer and wider leaves, has unmottled leaves, has longer trichomes on the leaf blade edge at the collar, with those trichomes placed further away from the collar, and has sheath trichomes whereas the new variety ‘Tift-23’ does not. ‘Tift-17’ is otherwise similar to the new variety ‘Tift-23’ in that it has similar abaxial leaf color, midrib color, has no trichomes on either leaf surface, is cold tolerant and disease resistant, and does not flower in late October.

### BRIEF DESCRIPTION OF THE FIGURES

The accompanying photographic illustration shows typical specimens in full colour of the foliage and fruit of the new variety Pennisetum ‘Tift-23’. The colors are as nearly true as is reasonably possible in a color representation of this type.

FIG. 1 is a photograph of the new variety Pennisetum ‘Tift-23’.

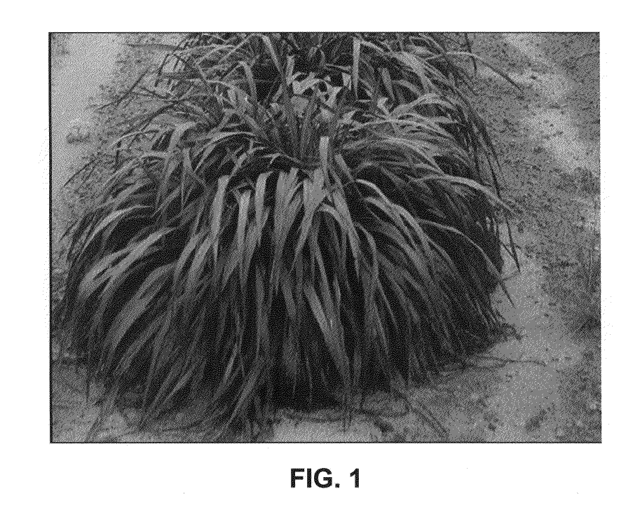

### BOTANICAL DESCRIPTION

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society, London, England.

The following observations, measurements and values describe plants grown in Tifton, Blairsville, or Griffin, Ga. During the growing of the plants, day temperatures ranges from approximately 50° F. to approximately 99° F. and night temperatures ranges from approximately 20° F. to approximately 70° F.

The new variety ‘Tift-23’ is a weak perennial at Tifton, Ga. (U.S.D.A. zone 8a). The new variety ‘Tift-23’ is pollen and seed sterile and does not produce seed heads at Tifton, Ga. in the field.

All data are from plants established as single stem propagules in mid-May, and rated in September through October. Plants were spaced at 2 meter centers. Color indicators are according to The Royal Horticultural Society (R.H.S.) Colour Chart, 5^{th} Edition.

Other data are as follows:
Mature plant height: Approximately 53-77 cm.
Diameter of plant canopy: Approximately 67-147 cm.
Diameter of base: Approximately 21-35 cm.
Leaf width: Approximately 18-22 mm.
Leaf length: Approximately 46-67 mm.
Adaxial leaf surface trichomes: None.
Abaxial leaf surface trichomes: None.
Leaf blade margin trichome length: Inconspicuous.
Leaf collar trichome length: Approximately 1 mm.
Leaf blade edge at collar trichome: Approximately 2 mm long for about 3 mm from collar
Sheath trichomes: None.
Number of tillers: Approximately 34-119.
Adaxial leaf color: About mottled Greyed-purple RHS 187D/137B.
Abaxial leaf color: About Greyed-purple RHS 187B.
Midrib color: About Greyed-purple RHS187B.
Inflorescence in late October: None.

What is claimed is:

1. A new and distinct variety of the Pennisetum plant named ‘Tift-23’, substantially as illustrated and described herein.
